(19) United States
(12) Patent Application Publication
(10) Pub. No.: US 2015/0307425 A1
(43) Pub. Date: Oct. 29, 2015

(54) RECAPTURE OF ETHANOL FROM AGING BARREL

(71) Applicant: Benjamin Cox, Georgetown, KY (US)

(72) Inventor: Benjamin Cox, Georgetown, KY (US)

(21) Appl. No.: 14/749,737

(22) Filed: Jun. 25, 2015

Related U.S. Application Data
(63) Continuation of application No. 13/899,746, filed on May 22, 2013, now Pat. No. 9,085,510.

(51) Int. Cl. C07C 29/78; B65B 3/04

(52) U.S. Cl. CPC C07C 29/78; B65B 3/04

(57) ABSTRACT

An enclosure that may be placed around a barrel of aging alcohol, wherein the enclosure may include a bottom, a net, a cooling coil, and a top, wherein the cooling coil may be positioned over the barrel such that ethanol vapor can be condensed onto the cooling coil and dripped onto the barrel. The cooling coil may also be cooled by cooling refrigerant below the condensation point of ethanol and pumping the refrigerant through the cooling coil. Alternatives can include positioning the cooling coil over a drip pan and may also include returning the condensed ethanol into the barrel via a hose.

### BACKGROUND

The present invention relates to aging alcohol, and more particularly to recapture of ethanol vapor during aging.

During the production of many drinking alcohols, such as whiskey, the alcohol is aged in a wooden barrel. However, these barrels are permeable to the liquid constituents of the alcohol, such as ethanol. In fact, some alcohol evaporates from the wooden barrel, resulting in decreased volume of alcohol as the aging process continues. This loss in volume is affectionately referred to as the “angel's share” in the industry.

### SUMMARY

Embodiments of the present invention relate to an enclosure including a bottom, a net, a cooling coil, and a top, wherein the bottom includes a rigid support structure that is impervious to ethanol vapor, wherein a width of the bottom is at least as wide as the belly of a barrel, wherein a length of the bottom is at least as long as the length of the barrel, wherein the length of the top is at least as long as the length of the barrel, wherein the width of the top is at least as wide as the width of the barrel, wherein the top comprises a material that is impervious to ethanol vapor, wherein the net extends orthogonally from the top for at least the distance of a widest belly diameter of the barrel, wherein the net is impervious to ethanol vapor, and wherein the cooling coil is positioned over the barrel.

Additional embodiments of the present invention relate to a method of condensing ethanol including placing a barrel containing alcohol within an enclosure, wherein the enclosure is impervious to ethanol vapor, and placing a cooling coil within the enclosure.

An enclosure including a bottom, a net, a cooling coil, and a top, wherein the bottom includes a rigid support structure that is impervious to ethanol vapor, wherein a width of the bottom is at least as twice as wide as the largest belly diameter of a barrel, wherein a length of the bottom is at least as long as the length of the barrel, wherein the length of the top is at least as long as the length of the barrel, wherein the width of the top is at least twice as wide as the largest belly diameter of the barrel, wherein the top comprises a material that is impervious to ethanol vapor, wherein the net extends orthogonally from the top for at least the distance of the largest belly diameter of the barrel, wherein the net is impervious to ethanol vapor, wherein the cooling coil is positioned over the barrel.

### BRIEF DESCRIPTION OF THE DRAWINGS

FIG. 1A illustrates a diagram of a single-barrel enclosure around a wooden barrel containing aging alcohol in accordance with the principles of the present invention.

FIG. 1B illustrates a side-view of the enclosure of FIG. 1A wherein a cooling coil comprises a rise from the center to the remainder of the cooling coil.

FIG. 2 illustrates a diagram of multiple single-barrel enclosures around respective wooden barrels containing aging alcohol in accordance with the principles of the present invention.

FIG. 3 illustrates a diagram of a multi-barrel enclosure around respective wooden barrels containing aging alcohol in accordance with the principles of the present invention.

FIG. 4 illustrates a block-level diagram of an example method to recapture of ethanol vapor in an enclosure in accordance with the principles of the present invention.

FIG. 5 depicts a top-down view of a representation of the configuration of a barrel, a net, and a cooling coil according to the principles of the present invention.

FIG. 6 depicts a side view of an arrangement of a cooling coil, a drip pan, a reinsertion hose, a bung hole, and a barrel in accordance with the principles of the present invention.

FIG. 7 depicts a block-level diagram of a controller in accordance with the principles of the present invention.

FIG. 8 illustrates an embodiment of the enclosure comprising an atmospheric balancing tube.

### DETAILED DESCRIPTION

The detailed description set forth below in connection with the appended drawings is intended as a description of various embodiments of the invention and is not intended to represent the only embodiments in which the invention may be practiced. The detailed description includes specific details for the purpose of providing a thorough understanding of the invention. However, it will be apparent to those skilled in the art that the invention may be practiced without these specific details. In some instances, well known structures and components are shown in block diagram form in order to avoid obscuring the concepts of the invention.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. Aspects of the invention were chosen and described in order to best explain the principles of the invention and the practical application, and to enable others of ordinary skill in the art to understand the invention for various embodiments with various modifications as are suited to the particular use contemplated.

During production of many alcoholic drinks, raw un-aged distillate is produced from fermented mashes. This distillate, affectionately referred to as “white dog” in the bourbon industry, comprises both ethanol and water and can be consumed immediately. However, many distillers age the distillate in barrels to alter the character of the distillate. The aging process occurs over a number of years, in which the barrels may be warmed and cooled. The wood pores of the warmed barrels expand and absorb alcohol, whereas the cooled barrel wood pores contract and force much of the alcohol back into the interior of the barrel. This interaction with the wood allows the aging alcohol to dissolve constituents of the wooden barrel. The dissolved constituents become congeners of the alcohol and may alter the smell, taste, burn, and mouthfeel of the alcohol. Further interactions with oxygen inside the barrel may also alter the character of the alcohol eventually bottled.

However, ethanol and water may evaporate and escape the barrel during the aging process. This evaporation causes a loss in volume that many refer to as the “angel's share.” The angel's share varies based on the climate in which the alcohol is aged and may be affected by humidity and temperature. Accordingly, the typical angel's share is about two percent volume per year and may be as high as three percent per year in some geographic locations.

Prevention of loss of the angel's share by wrapping a barrel with a gas-impervious material may slow or alter the aging process. This may be due to the sequestration of oxygen, necessary for the aging process, from the aging alcohol. Thus, a structure for trapping alcohol vapor while allowing sufficient oxygen for the aging process may be desirable.

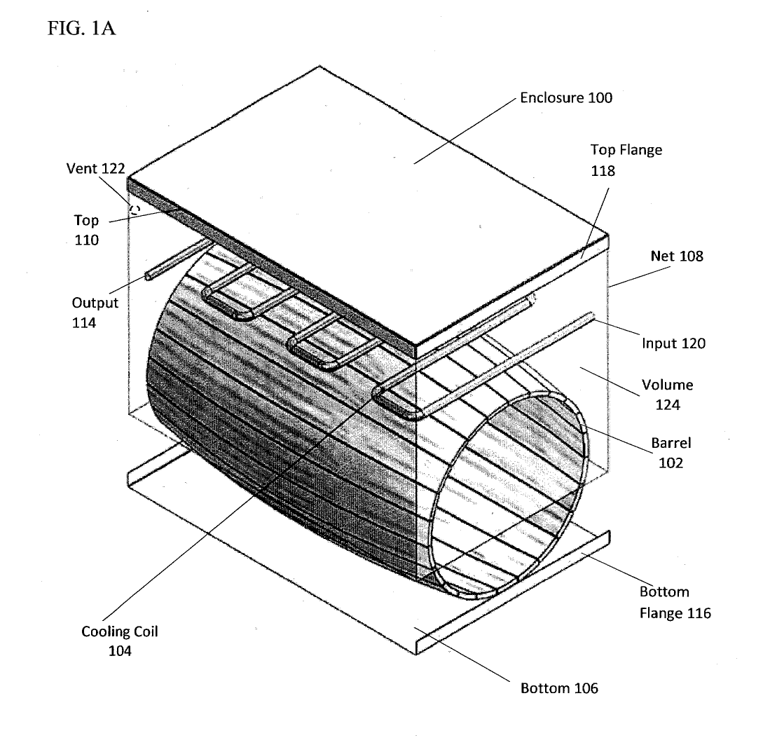

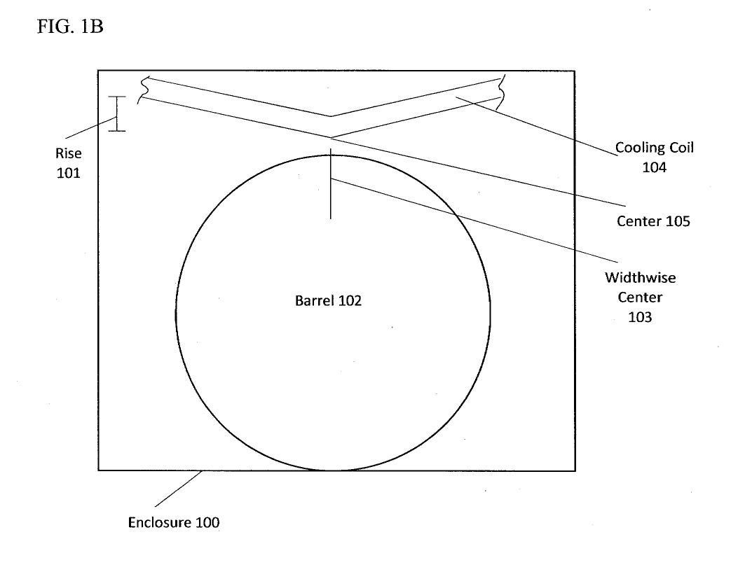

FIG. 1A illustrates a diagram of an enclosure 100 around a wooden barrel 102 containing aging alcohol in accordance with the principles of the present invention. The wooden barrel 102 may be made of oak, cedar, or any other wood suitable for aging alcohol. Furthermore, the barrel 102 may be caramelized or charred. A standard burgundy wine barrel could be around 60 cm (23.6 inches) in head diameter, 72 cm (28.3 inches) in belly diameter, and 88 cm (34.6 inches) in length. Barrels may have a tapered body having a smallest diameter at each head and a largest diameter at the center, or the “belly.” Barrel widths for many other distilled spirits can vary from between a smallest belly diameter of 63.5 cm (25 inches) and a largest belly diameter of 71.12 cm (28 inches). A small barrel may have a 53.34 cm (21 inch) head diameter and a 58.42 cm (23 inch) belly diameter. Length may not necessarily vary between sizes of barrel. A whiskey barrel may be large enough to store 200 liters (53 gallons) and may have a 71.12 cm (28 inches) belly diameter, a 66.04 cm (26 inches) head diameter, and a 91.44 cm (36 inches) length. Wine barrels may also be found in 246 liter (65 gallon), 265 Liter (70 gallon), and 454.2 liter (120 gallon) volumes. Of course, barrels having other lengths, head diameters, and belly diameters exist. Furthermore, use of these barrels or sizing the enclosure to fit these barrels is within the scope of the present invention.

The size of the barrel 102 may be used to determine the size of the enclosure 100, because the enclosure 100 may enclose the barrel 102. The enclosure 100 may comprise a bottom 106, a net 108, a top 110, and a cooling coil 104. The term “net” as used herein, and in the attached claims, is intended to encompass a material that is substantially impervious to ethanol vapors rather than a woven or knotted open mesh fabric.

Embodiments of the enclosure 100 include a single-barrel embodiment. The enclosure 100 may be sized to have a large volume 124 between the enclosure 100 and the barrel 102. The volume 124 may comprise the space between the outer surface of the barrel 102 and the inner surface of the enclosure 100. For example, the volume 124 may comprise one cubic meter of space in which the evaporated ethanol vapor may collect within the enclosure 100 and outside the barrel 102. Other embodiments may contain half of a cubic meter of volume 124 between the enclosure 100 and the barrel 102. This space may allow the presence of sufficient oxygen for the aging process. In other embodiments, the enclosure 100 may be sized for a small volume 124 such that the belly of the barrel 102 touches the bottom 106 and two respective sides of the net 208. Furthermore, both heads of the barrel 102 may contact respective portions of respective sides of the net 208. Additional embodiments include the enclosure 100 sized for a small volume 124 such that the head of the barrel 102 touches the bottom 106 and the belly of the barrel 102 touches four respective sides of the net 208. Additionally, the top 110 may be positioned such that the cooling coil 104 is one centimeter from the top 110. In other embodiments, the top 110 may be positioned such that the cooling coil 104 is one millimeter from the top 110. In some embodiments, the cooling coil 104 does not touch the barrel 102. Therefore, the cooling coil 104 may be one centimeter from the barrel 102. In other embodiments the cooling coil 104 may be one millimeter from the barrel. This small volume sizing may be useful for close packing of barrels 102 within single-barrel embodiments of the enclosure 100 within a warehouse.

Other embodiments, described later, include multi-barrel enclosures 100 such that multiple barrels 102 that may be placed within the same enclosure 100. Another example embodiment includes a plurality of barrels 102 may be stored on a rick and enclosed within a single enclosure 100. In a multi-barrel embodiment, the net 108 may contact respective portions of the respective bellies of the two outer barrels 102 in the enclosure 100. Additionally, all the barrels 102 within the enclosure 100 may rest on the bottom 106. The heads of the respective barrels 102 may also contact respective portions of the net 108. Furthermore, the spacing between the top 110 and the respective barrels 102 and the spacing between the respective cooling coils 104 and the respective barrels 102 may be similar to corresponding respective distances of the single-barrel embodiment.

The enclosure 100 may be impervious to ethanol vapor. Therefore, the evaporating ethanol may become trapped within the volume 124 of the enclosure 100. In other embodiments, the enclosure 100 may slow down the escape of ethanol vapor rather than completely stop the escape of the vapor.

The bottom 106 may comprise any structure of sufficient strength to support a barrel 102 full of aging alcohol. In some embodiments, the full barrel 102 can weigh 500 pounds or more. The bottom 106 may also comprise any shape such that the barrel 102 fits on the bottom 106. For embodiments sized to the belly of the barrel 102 on the bottom 106, the width of the bottom 106 may be greater than or equal to the largest belly outer surface diameter of the barrel 102. Furthermore, the length of the bottom 106 may be longer than or equal to the length of the barrel 102 measured from head to head. For embodiments sized to the head of the barrel 102 on the bottom 106, the width of the bottom 106 may be equal to or greater than the diameter of the head of the barrel 102. Furthermore, these embodiments may include a width of the bottom 106 that is as wide as or wider than the belly diameter of the barrel 102. Additionally, the length of the bottom 106 may be similar to the width of the bottom 106. For example, a flat rectangular bottom 106 may suffice for resting a barrel 102 of aging whiskey.

One or more edges of the bottom 106 may be attached to or integrally formed with a bottom flange 116. In some embodiments, the flange 116 may be sufficient to retain any pooled condensate that may drip over the barrel 102. For example, the flange 116 may extend orthogonally away from the bottom 106 at a height of one centimeter or greater.

The bottom 106 may be formed of any structure sufficient for retaining the weight of the full barrel 102. The material may also be corrosion and rust resistance, such that the taste of the aging alcohol is not altered by contact of ethanol with the bottom 106. Example materials may include aluminum and stainless steel.

The top 110 may comprise any structure of sufficient strength to support a hanging cooling coil 104 full of refrigerant. The top 110 may also comprise any shape such that the barrel 102 fits within the boundaries of the top 110 when viewed top-down. For example, the width of the top 110 may be greater than or equal to the largest belly outer surface diameter of the barrel 102. Furthermore, the length of the top 110 may be longer than the outer length of the barrel 102 measured from head to head. In embodiments sized to the head of the barrel 102 on the bottom 106, the length and width of the top 110 may be equal to or larger than the diameter of the head of the barrel 102. Alternatively, the length and width of the top 110 could be equal to or larger than the belly diameter of the barrel 102. For example, a flat rectangular top may suffice for hanging a cooling coil 104 over a barrel of aging alcohol.

One or more edges of the top 110 may be attached to or integrally formed with a top flange 118. In some embodiments, the flange 118 may be sufficient to hang the net 108 around the barrel 102. For example, the flange 118 may extend orthogonally away from the top 110 at a height of one centimeter or greater. The net 108 may be hung from the flange 118 through attachment to or integral formation with the flange 118. Alternatively, the net 108 may be directly hung from an edge or a face of the top 110.

The top 110 may be formed of any structure sufficient for retaining the weight of the full cooling coils 104 suspended from the top 110. The material of the top 110 may also be corrosion and rust resistance, such that the taste of the aging alcohol is not altered by contact of ethanol with the top 110. Example materials may include aluminum and stainless steel.

The net 108 may be attached to or integrally formed with the top 110. This attachment may occur at the edge of the flange 118 opposite the top 110, on the face of the top 110 facing the barrel, or at the edge of the top 110. The net 108 may extend orthogonally away from the top 110 such that a perimeter is formed. This perimeter may be sized such that the perimeter will fit around the barrel 102. Furthermore, the net 108 may extend such that the barrel 102 is surrounded by the net 108 in the orthogonal distance from the top 110. For example, the net 108 may extend at least the distance between the top 110 and the cooling coil 104 plus the distance between the cooling coil 104 and the barrel 102 plus the diameter of the barrel 102. In embodiments wherein the enclosure 100 is sized to a barrel 102 standing on the head, the net 108 may extend the distance between the top 110 and the cooling coil 104 plus the distance between the cooling 104 and the barrel 102 plus the height of the barrel 102. Furthermore, the net may comprise an unbroken perimeter, a broken perimeter, or a breakable perimeter. In the example of FIG. 1A, the perimeter of the net 108 is rectangular in shape. For example, the corners of the perimeter may be integrally formed or may be joined by fasteners, such as Velcro, snaps, pins, adhesives, etc. In embodiments having a breakable perimeter, the fasteners may be reattachable fasteners, such that the net 108 can be opened and then resealed. Opening the perimeter of the net 108 may be useful to reach the barrel 102, such as during an audit or for taking a sample during aging. Alternatively, the breakable net 108 could be broken to refresh the oxygen and then resealed. In embodiments having a breakable perimeter, the fasteners may create a seal such that ethanol vapor does not leak through the attachment of the perimeter. In embodiments having a broken perimeter, a hole may exist in the net 108, such as vent 122.

The net 108 may be formed from any material sufficient to keep ethanol vapor from escaping the volume 124. Example materials include mylar, polyester films, or other plastics formed from Polyethylene Terephthalate. Additionally, the net 108 may be pervious to oxygen while being impervious to ethanol. The net 108 may extend orthogonally away from the top 110 to the bottom 106. The net 108 may be integrally formed with the bottom 106. Alternatively, the net 108 may simply contact the bottom 106 or the bottom flange 116 without attachment. Further embodiments include a removable attachment between the bottom 106 and the net 108. Examples may include Velcro, snaps, pins, adhesives, etc. Embodiments having no attachment or removable attachment may be useful for raising the net 108 for a barrel audit or for taking a sample from the barrel 102 during the aging process.

The vent 122 may comprise a hole, a slit, or a tube allowing air flow through the net 108. The vent 122 may be placed such that the atmospheric pressure within the enclosure 100 is equal to the pressure outside the enclosure 100 and such that release of ethanol vapor is minimized. The vent 122 may comprise a tube placed at the bottom 106 and extending to the top 110 of the enclosure 100. For example, a first end of the vent tube may extend through the bottom 106 or through the bottom of the net 108, and a second end of the vent tube may open into the volume 124 near the top 110. This tube may comprise plastic, rubber, etc. As another example, the space between the cooling coil 104 and the top 110 may have minimal ethanol vapor. Therefore, the vent 122 may be placed in the net 108 such that the vent 122 is between the cooling coil 104 and the top 110 when viewed from a side view of the enclosure 100. Alternatively, the vent 122 may be placed within the top quarter of the orthogonal distance of the net 108. In other embodiments, the vent 122 may be placed in the bottom quarter of the orthogonal distance of the net 108. Additionally, multiple vents 122 may be placed in the net to provide optimal oxygen intake into the volume 124 while minimizing ethanol vapor escape. The vent 122 may be 1 mm in diameter.

The cooling coil 104 may comprise a pipe having a surface area, a length, an input 120, and an output 114. In this manner, the cooling coil 104 may allow the transfer of refrigerant, such as water, through the cooling coil 104. The refrigerant may comprise water or any other fluid sufficient for altering or maintaining the temperature of the cooling coil 104. Examples include air, oxygen, nitrogen, aqueous solutions, ammonia, sulfur dioxide, propane, chlorofluorocarbons, etc. When to describe the fluid in the container 206 or the cooling coil 104, the term “water” is intended to include refrigerant. The cooling coil 104 may also be doubled-back over the barrel 102 one or more times as depicted in FIG. 1A. Each straight section of the body of the cooling coil 104 may have a length substantially equal to or less than the width of the barrel 102 before joining a u-joint to double-back the cooling coil 104 over the barrel 102. This pattern may increase the surface area of the cooling coil 104 that is directly over the barrel 102 such that ethanol evaporating from the barrel 102 may condense on the cooling coil 104 and may drip directly back onto the barrel 102. Furthermore, the majority or all of the length of the cooling coil 104 may be positioned over the barrel 102 such that condensate is dripped onto the barrel 102. In these embodiments, the coiled portions of the cooling coil 104 may be positioned such that condensate drips directly onto the barrel 102. However, the output 114 and the input 120 may extend beyond the net 108, and therefore these small portions of the cooling coil 104 may overhang the barrel 102. In one embodiment, the cooling coil 104 may double back at least three times, may comprise a diameter of 2 cm, and may be spaced 5 cm or more from the neighboring doubled-back portions of the cooling coil 104. In another embodiment, the cooling coil 104 may be doubled-back 5 times and may be spaced 15 cm from the neighboring doubled-back portions.

FIG. 1B illustrates a side-view of the enclosure of FIG. 1A wherein a cooling coil comprises a rise from the center to the remainder of the cooling coil. In this embodiment, the cooling coil 104 may be shaped such that the center 105 of the cooling coil 104 hangs lower than the remainder of the cooling coil 104. Furthermore, the center 105 may hang over the widthwise center 103 of the barrel 102. The rise 101 may be the distance between the outer edges and the center 105 of the cooling coil. The rise 101 may cause a slope along the surface of the cooling coil whereby condensate on the surface of the cooling coil 104 may run to the center 105 and may drip onto the barrel 102. Thus, the condensate may collect at the low point over the center 105 of the barrel 102 such that the condensate drips onto the widthwise center 103 of the barrel 102. In alternative embodiments, the cooling coil 104 may be tilted such that the condensate drips beside the barrel 102 and into a drip pan. For example a container could be used to catch the angel's share condensate for separate labeling and marketing as the angel's share.

Alternatively, condensate can be reinserted into the barrel 102. This reinsertion into the barrel 102 can be accomplished by dripping the condensate directly into an opening of the barrel 102. In other embodiments, the condensate can be collected in a drip pan, wherein the drip pan comprises a base and a perimeter extending orthogonally away from the base. The drip pan may be positioned such that the drip pan opens toward the cooling coil to receive dripped condensate. The contents of the drip pan may then be pumped or gravity fed through a reinsertion hose and into the barrel 102. A first end of the reinsertion hose may be connected with a bung hole of the barrel 102. For example, the first end of the reinsertion hose may be coupled with a cork shaped to fit the bung hole. Placing this cork in the bung hole may set the first end of the reinsertion hose open to the interior of the barrel 102. The cork and the first end of the reinsertion hose may be sealed by adhesive, tape, gel, silicone, caulk, etc. or could be integrally formed. A second end of the reinsertion hose may be connected with or placed in the drip pan. Thus, pumping the contents of the drip pan through the hose may return the condensate to the interior of the barrel 102.

The water, or refrigerant, running through the cooling coil 104 may regulate the temperature of the cooling coil 104. For example, the water may be cooled to 4.4° C. (40° F.). The material of the cooling coil 104 may be selected to retain the shape of the cooling coil 104, to support the weight of the cooling coil 104 filled with water suspended from the top 110, to have high thermal conductivity, and to have low thermal emissivity. An example material may include copper, aluminum, stainless steel, etc. The cooling coil 104 may therefore be cooled by the water to 4.4° C. Thus, ethanol vapor that contacts the surface area of the cooling coil 104 may condense on the cooling coil 104. The low thermal emissivity of the cooling coil 104 may allow for condensing ethanol with minimized temperature regulation of the volume 124. Furthermore, the enclosure 100 may allow transfer of heat between the volume 124 and the atmosphere outside the enclosure 100. Therefore, the wood of the barrel 102 may undergo expansion and contraction due to ambient temperature changes outside the enclosure 100.

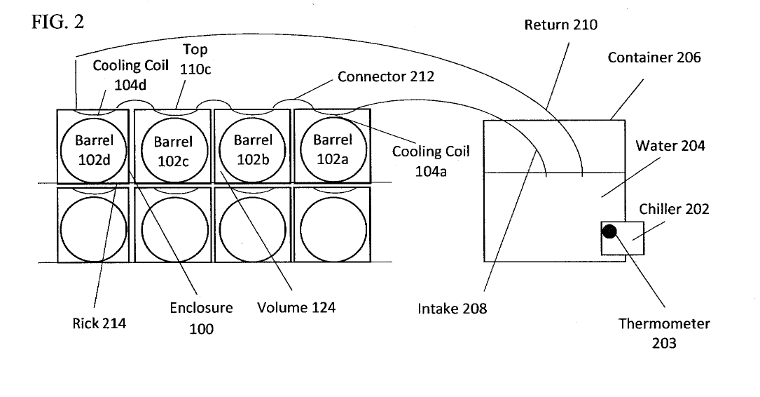

FIG. 2 illustrates a diagram of multiple single-barrel enclosures around respective wooden barrels containing aging alcohol in accordance with the principles of the present invention. The enclosures 100 may be placed on a rick 214 in an aging storehouse. As with the embodiment depicted, an intake 208 may pump chilled water 204 from the container 206 to a first cooling coil 104. One or more respective connectors 212 may connect respective cooling coils 104 and therefore chilled water 204 may be transferred through the respective cooling coils 104 of several enclosures 100 in series. A return 210 may be connected with the output 114 of the last respective cooling coil of the series. This return 210 may receive the water 204 and may return the water 204 to the container 206.

The input 120 and the output 114 may be positioned within the net 108. Thus, an intake 208 connected with the input 120 may extend through the net 108, and a return 210 connected with the output 114 may extend through the net 108. Alternatively, the cooling coil 104 may extend through the net 108 such that the input 120 and the output 114 are outside the net 108. The intersection of the intake 208 and net 108, return 210 and net 108, or cooling coil 104 and net 108 may comprise a vent 122 sized to regulate oxygen and ethanol vapor exchange between the volume 124 and ambient atmosphere. Alternatively, these respective intersections may comprise an ethanol impervious attachment between the respective elements. Such an attachment could comprise caulk, putty, tape, silicone, integral formation, etc.

An intake 208 connected with the input 120 may extend through the top 110a over a first barrel 102a on the rick 214 and a return 210 connected with the output 114 may extend through the top 110d over a last barrel 102d. Each intervening connection between the respective cooling coils 104 may occur between the respective nets 108 of the respective enclosures 100. Alternatively, the respective cooling coils 104 for each respective barrel 102 may extend through each respective top 110 such that the respective inputs 120 and respective outputs 114 are outside the top 110. The respective cooling coils 104 may be joined by respective connectors 212, wherein a first end of each respective connector is connected with the output 114 of a cooling coil 104 and a second end of each respective connector 212 is connected with the input 120 of a cooling coil 104. The intersection of the intake and top 110, return and top 110, or cooling coil 104 and top 110 may comprise a vent 122 sized to regulate oxygen and ethanol vapor exchange between the volume 124 and ambient atmosphere. Alternatively, these respective intersections may comprise an ethanol impervious attachment between the respective elements. Such an attachment could comprise caulk, putty, tape, integral formation, etc.

Returning to FIG. 2, the intake 208 may be any structure suitable for transporting water 204 from the container 206 to the cooling coil 104. Example structures includes pipes, hoses, etc. The intake 208 may be positioned such that a first end of the intake 208 is submerged in the water 204 and that a second end of the intake 208 is connected to the input 120. Furthermore, the material of the intake 208 may comprise materials that retain structural integrity during contact with water. Example materials may include copper, steel, polyvinyl chloride (PVC), rubber, etc. However, in some embodiments, the intake 208 may also insulate the chilled water 204 within the intake 208. In these embodiments, the intake 208 may comprise insulators, such as rubber, fiberglass, foam, etc. Furthermore, the intake 208 may comprise a pump at the end of the intake 208 submerged in the container 206. This pump may provide the water pressure and volume sufficient to cycle chilled water through the cooling coil 104.

The connector 212 may comprise any structure suitable for transporting water from one cooling coil 104 to an adjacent cooling coil 104. Example structures includes pipes, hoses, etc. Furthermore, the material of the connector 212 may comprise materials that retain structural integrity during contact with water. Example materials may include copper, steel, polyvinyl chloride (PVC), rubber, etc. However, in some embodiments, the connector 212 may also insulate the chilled water 204 within the connector 212. In these embodiments, the connector 212 may comprise insulators, such as rubber, fiberglass, foam, etc. The connector 212 may connect the output 114 of a first cooling coil 104 with the input 120 of a second cooling coil 104 such that water may be transferred from the first cooling coil 104 to the second cooling coil 104.

The return 210 may comprise any structure suitable for transporting water from the cooling coil 104 to the container 206. Example structures includes pipes, hoses, etc. The return 210 may be connected to the output 114 of the cooling coil 104 at a first end of the return 210. A second end of the return 210 may be within the container 206 or connected to the container 206 such that water 204 may be pumped into the container 206 from the return 210. Furthermore, the material of the return 210 may comprise materials that retain structural integrity during contact with water. Example materials may include copper, steel, polyvinyl chloride (PVC), rubber, etc. However, in some embodiments, the return 210 may also insulate the chilled water 204 within the return 210. In these embodiments, the return 210 may comprise insulators, such as rubber, fiberglass, foam, etc.

The container 206 may comprise any size and shape sufficient for retaining a volume of water 204 for cooling the cooling coils 104 of all the enclosures 100. For example, the container 206 could be a cylinder, a cube, a pyramid, etc. Furthermore, the material of the container 206 may comprise materials that retain structural integrity during contact with water. Example materials may include copper, steel, polyvinyl chloride (PVC), rubber, etc. Furthermore, the container 206 may insulate the water within the container 206. Therefore, the container 206 may comprise insulators, such as rubber, fiberglass, foam, etc.

The chiller 202 may be in fluid communication with the container 206. As depicted, the chiller 202 can be integrally formed with the container 206 and can be partially within and partially outside the container 206. However, the chiller 202 could be placed entirely within the container 206 or entirely outside the container 206. The chiller 202 may refrigerate or cool the temperature of the water 204 to a temperature suitable for condensing ethanol vapor (e.g. 4.4° C.). This cooling can be performed by gas expansion, heat pump, ice, magnetic cooling, laser cooling, or any other modern cooling method. The return 210 and the intake 208 may intersect the container 206. At these respective intersections, the return 210 and the container 206 may be joined or integrally formed and the intake 208 and the container 206 may be joined or integrally formed. These respective intersections may also comprise sealants to prevent the evaporation of water vapor from the container. Such sealants could include caulk, silicone, gels, tape, etc. Furthermore, the respective intersections could also comprise an insulator to aid in keeping the water cooled. This insulator may be similar to the insulator that may be used for the container 206.

Water 204 may be placed in the container 206 and may be pumped through the intake 208, the cooling coils 104, the connectors 212 (if present), and the return 210. The water 204 may contact the chiller 202, wherein the chiller 202 may cool the temperature of the water 204. The water 204 may comprise water, aqueous solutions, or even organic solutions. However, the water 204 may have a high specific heat, such as is found with water or aqueous solutions. As ethanol condenses on the cooling coil 104, the cooling coil 104 and the water therein may receive thermal energy. Thus, the temperature of the water may rise until the water is returned to the container 206. Cooler water within the container 206 may sink due to the increased density of the cooler water 204. Thus, the intake 208 may be positioned to pump out this cooler water at the bottom of the container 206.

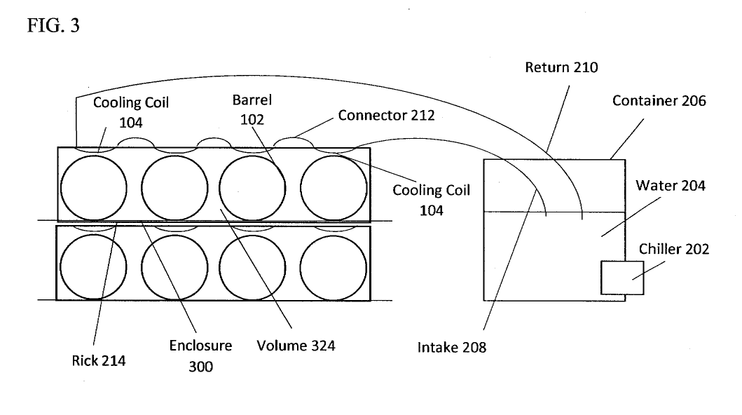

FIG. 3 illustrates a diagram of a multi-barrel enclosure 300 around respective wooden barrels containing aging alcohol in accordance with the principles of the present invention. As depicted, the respective cooling coils 104 may be positioned directly over the respective barrels 102. Thus, the cooling coils 104 may be connected outside the enclosure 100 by connectors 212 in order to reduce surface area of the cooling coils that could drip between the barrels 102. Alternatively, the cooling coils 104 could be directly connected within the enclosure 100. In this embodiment, the sections of cooling coil 104 overhanging the barrels 102 could be insulated to prevent condensation of ethanol where the condensate would not drip onto the barrels 102. Alternatively, portions of the respective cooling coils 104 that are not directly over a barrel 102 may be raised such that any condensate may run down to a lower point on the cooling coil 104 and then drip onto the barrel 102.

The multi-barrel enclosure 100 may comprise an open interior and therefore a continuous volume 324 may be formed around the respective barrels 102. As shown, the barrels 102 may be placed with space between the barrels 102. However, the barrels 102 may be placed such that the respective bellies of the barrels 102 touch. Alternate multi-barrel embodiments of the present invention also exist, wherein the condensate is collected in a drip pan rather than dripped back onto the barrels 102.

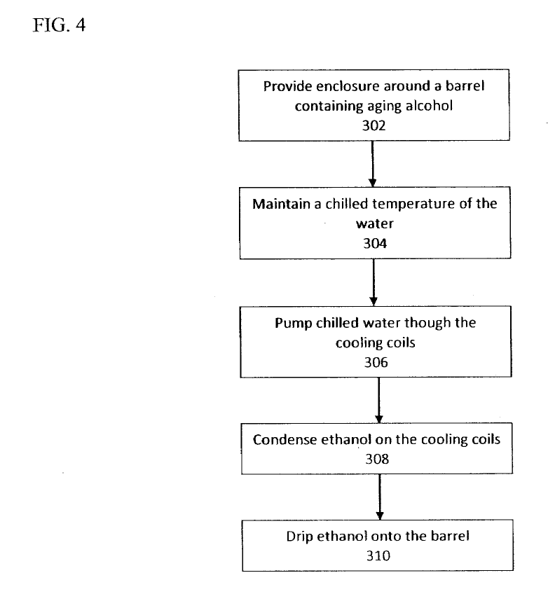

FIG. 4 illustrates a block-level diagram of a method to recapture of ethanol vapor within an enclosure 100 in accordance with the principles of the present invention. In step, 302 an enclosure may be provided around the barrel 102 containing aging alcohol. This enclosure may capture the ethanol vapor and water vapor that evaporated from the barrel 102. In step 304, water may be maintained at a chilled temperature, such as within three degrees of 4.4° C. The chilled water may be pumped through respective cooling coils located above a barrel 102, in step 306. Thus, the temperature of the cooling coils 104 may be lowered such that ethanol within an enclosure surrounding the barrel 102 may condense onto the cooling coils 104.

Of course, a chiller for chilling the water or refrigerant can include a thermometer positioned to measure the temperature of the water by thermal communication between the thermometer and the water. Alternatively, the thermometer may be placed in a container holding the water and may be in electronic communication with the chiller or a controller. The chiller can also start cooling when a predetermined high temperature is reached by the thermometer. The predetermined high temperature should be below the condensation temperature of ethanol (e.g. below 78.37° C.). However, the predetermined high temperature may be lower, such as 4.4° C. The chiller may stop cooling when a predetermined low temperature is reached such that the water is not frozen in the container and such that neither ethanol nor water is frozen onto the cooling coils 104. Thus, the predetermined low temperature could be 0° C. or higher. In this manner, the temperature of the cooling coils 104 in step 306 may be regulated.

In step 306, the chilled water may be pumped through the cooling coil 104. Thus, the temperature of the cooling coil 104 may be reduced by thermal conduction of heat from the cooling coil 104 to the chilled water. In step 308, the ethanol may be condensed onto the cooling coils 104. The rate of condensation may be controlled by the surface area of the cooling coil 104 over the barrel 102. The surface area of the cooling coil 104 may be determined by the number of times the cooling coil 104 doubles back over the barrel 102 and/or the diameter of the cooling coil pipe. For example, a desirable rate of condensation may not necessarily create pooled liquid within the enclosure 100. In one embodiment, the cooling coil 104 has a diameter of 2.54 cm and doubles back over the barrel 102 five times.

In step 310, the condensate may be dripped back onto the barrel 102. The shape of the cooling coils 104 may be such that gravity causes the condensate to collect over a portion of the barrel 102, such as the widthwise midline. The collection of condensate may result in drips due to the adhesion and weight of the condensate droplets overpowering the cohesion of the droplet to the surface of the cooling coil 104. The condensate may alternatively be dripped onto a different portion of the barrel 102. The condensate could be collected in a pan and labeled and sold as the “angel's share” for marketing purposes.

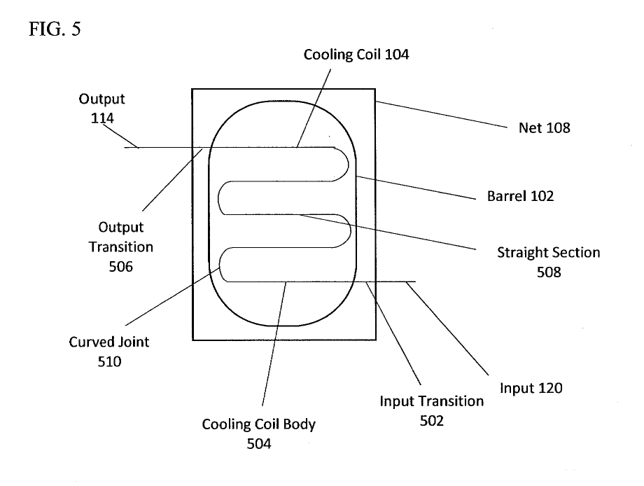

FIG. 5 depicts a top-down view of a representation of the configuration of the barrel 102, the net 108, and the cooling coil 104 of FIG. 1A. The cooling coil 104 may be positioned over the barrel 102. As depicted, the cooling coil body 504 may comprise multiple sections of straight section 508 pipe as well as the curved joint 510 pipe whereby the cooling coil 104 doubles back over the barrel 102. Therefore, the body 504 may be said to be over the barrel 102. As can be seen, the body 504 may overlap the barrel 102 when viewed from top-down. Thus, condensate dripping from the body 504 may fall onto the barrel 102. Each straight section 508 of the body 504 of the cooling coil 104 may have a length substantially equal to or less than the width of the barrel 102 before joining a u-joint to double-back the cooling coil 104 over the barrel 102. In alternative embodiments, each straight section 508 may have a length substantially equal to or less than the head of the barrel 102. An input transition 502 may be formed such that the cooling coil 104 extends beyond the net 108. The input transition 502 may be insulated to prevent condensation. Alternatively, the input transition 502 may be raised such that condensate runs to the body 504. The output transition 506 may be formed such that the cooling coil 104 extends beyond the net 108. The output transition 506 may be insulated to prevent condensation. Alternatively, the output transition 506 may be raised such that condensate runs to the body 504.

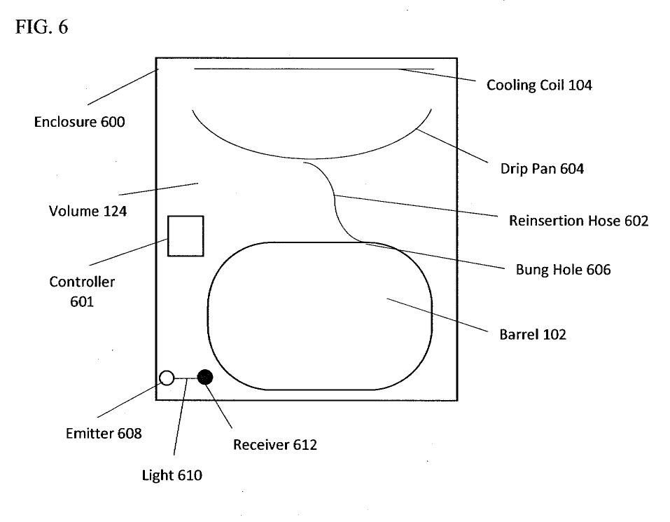

FIG. 6 depicts a side view of an arrangement of the cooling coil 104, a drip pan 604, a reinsertion hose 602, a bung hole 606, and the barrel 102 in accordance with the principles of the present invention. The drip pan 604 may be positioned under the cooling coil 104, the cooling coil body 504, or a portion of the cooling coil 104. Furthermore, part of the cooling coil 104 may be curved or angled to direct the condensate to drip into the drip pan 604. The drip pan 604 may comprise a hole fitted to a second end of the reinsertion hose 602. Alternatively, the drip pan 604 may be integrally formed with the reinsertion hose 602. In other embodiments, the second end of the reinsertion hose 602 may be placed in the drip pan over the perimeter of the drip pan 604. A first end of the reinsertion hose 602 may be connected to or integrally formed with the barrel 102, such as at the bung hole 606. In this manner, gravity may cause condensate to drip back into the barrel 102. Alternatively, a pump could be placed in the drip pan 604 and used to pump condensate from the drip pan 604 through the reinsertion hose 602 and into the barrel 102. The drip pan 604 may comprise any material that can retain shape in the presence of water and ethanol. Example materials include rubber, plastics, metals, etc. Additionally, the reinsertion hose 602 may comprise any material that can retain shape in the presence of water and ethanol. Example materials include rubber, plastics, metals, etc.

An emitter 608 and a receiver 612 may also be included within the volume 124 of the enclosure 100. The emitter 608 and the receiver 612 may be in electronic communication with the controller 601. The emitter 608 and the receiver 612 may be placed apart at a known distance, such as one centimeter. Furthermore, the emitter 608 and the receiver 612 may be positioned such that light 610 emitted by the emitter 608 may be received by the receiver 612. The emitter 608 may comprise a light source. In some embodiments, the emitter 608 may allow selection of a single or a few wavelengths of light 610. Furthermore, the receiver 612 may receive transmittance of a single wavelength or a spectrum of wavelengths of light 610. The wavelength may be selected such that the absorbance and scattering of ethanol or water or the mixture of the ethanol/water vapor may be used to determine concentration in the air. By emitting light 610 via emitter 608 through a known distance of the volume 124 to the receiver 612, the transmittance (e.g. the inverse of absorbance and scattering) may be used to determine the concentration of ethanol vapor and/or water vapor in the volume 124. For example, a control may be performed wherein the transmittance is measured with no ethanol/water vapor, with saturation of ethanol/water vapor in the volume 124, and at each five percent concentration interval between no vapor and saturation. Thus, the measured transmittance at the receiver 612 may be correlated with a concentration based on the control. Then, the chilled water could pumped into the cooling coil 104 when the transmittance falls below a predetermined level (indicating high vapor concentration), such as seventy-five percent or fifty percent of vapor saturation. Additionally, the pump may prevent chilled water from entering the cooling coil 104 if the transmittance rises above a predetermined level (indicating low vapor concentration), such as twenty-five percent or fifty percent vapor saturation.

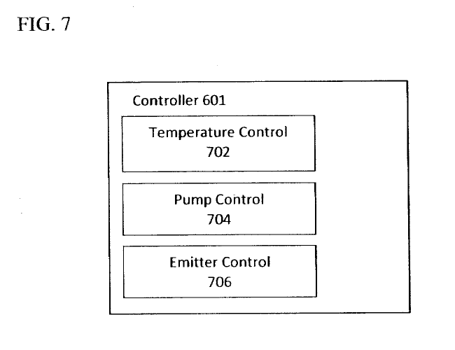

FIG. 7 depicts a block-level diagram of a controller in accordance with the principles of the present invention. The controller 700 may be in electronic communication with components of the enclosure 100, sensors, or other electronic components. For example, the controller 700 may be in electronic communication with one or more of the thermometer 203, the chiller 202, the pump of the intake 208, the emitter 608, and the receiver 612. The controller 700 may receive a temperature from the thermometer 203. When the temperature is high, such as 78.37° C., the controller 700 may activate the chiller 202 to cool the water 204. When the temperature is low, such as the near the freezing point of the water (e.g. 0° C.), the controller 700 may deactivate the chiller 202. In other embodiments, the controller 700 may activate and deactivate the chiller 202 in order to maintain the temperature of the water within three degrees of 4.4° C.

The controller 700 may activate and deactivate the emitter 608 and may alter the wavelength emitted by the emitter 608. The controller 700 may further receive transmitted light from the receiver 612. Based on this transmittance, the controller 700 may activate or deactivate the pump of the intake 208. For example, the ethanol/water vapor concentration may be desired to remain between 25 percent and 75 percent of saturation concentration of the volume 124. Thus, when a transmittance corresponding to 75 percent saturation is received, the pump may be activated. This may result in chilled water cooling the cooling coil 104 and condensation of the vapor on the cooling coil 104. Additionally, a transmittance corresponding to 25 percent saturation may be received by the controller 700, and then the controller 700 may deactivate the pump. This may result in warming of the cooling coils 104 and may allow the ethanol/water vapor concentration to increase in the volume 124. The cooling coil 104 may also be prevented from cooling the volume 124 and the barrel 102. Thus, another thermometer could be configured to measure the temperature of the volume 124 and could be configured to communicate with the controller 700. The controller 700 could ensure that the barrel 102 is allow to heat and cool for expansion and contraction based on outside climate.

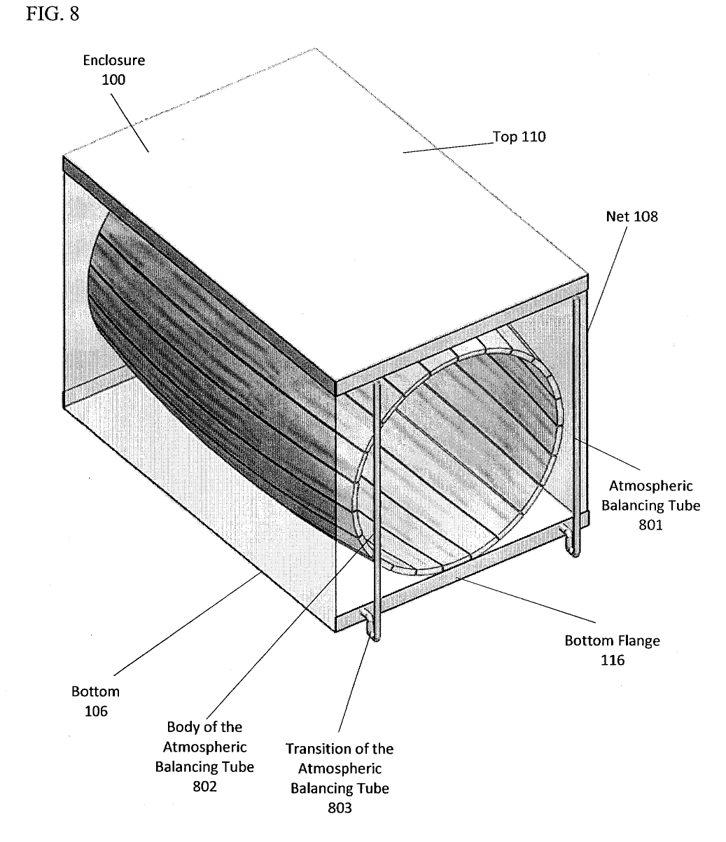

FIG. 8 illustrates an embodiment of the enclosure 100 comprising an atmospheric balancing tube 801. The atmospheric balancing tube 801 may comprise any structure sufficient to allow oxygen to enter the enclosure 100 while minimizing loss of ethanol vapor. Oxygen has a molecular weight of 32 g/mol. Oxygen can diffuse via Brownian motion. Thus, one example of the atmospheric balancing tube 801 includes a transition 803 that extends orthogonally from the bottom flange 116 outside the net 108. This atmospheric balancing tube 801 may fit a hole in the bottom flange 116 such that oxygen may transfer into the enclosure 100. The transition 803 may also comprise a curved pipe connected with the orthogonal pipe connected to a body of the atmospheric balancing tube 802. The body 802 may extend from the curved pipe up to or beyond the top 110. The diameter of the atmospheric balancing tube 801 may be sized to restrict the loss of ethanol vapor from within the enclosure 100. Thus, the diameter of the pipe of the balancing tube 801 may be 2.54 cm or less. The atmospheric balancing tube 801 may comprise any material that allows oxygen to pass through the atmospheric balancing tube 801, including PVC, metals, plastics, rubber, etc. In other embodiments, the transition 802 may comprise multiple curved joints such that the atmospheric balancing tube 801 extends orthogonally from the bottom flange 116, connects with a downward curved joint, extends to or beyond the bottom 106, connects with a curved joint, such as a U-joint, and then connects to the body of the atmospheric balancing tube 802 which extends to the top 110.

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a controller, a general purpose computer, a special purpose computer, or other programmable data processing apparatus to produce a machine, such that the instructions, which executed via the processor of the controller or other programmable data processing apparatus, create means for implementing the functions/acts specified in the flowchart and/or block diagram block or blocks.

These computer program instructions may also be stored in a computer readable medium that can direct the controller, the computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto the controller, a computer, other programmable data processing apparatus, or other devices to cause a series of operational steps to be performed on the controller, other programmable apparatus or other devices to produce a controller implemented process such that the instructions which execute on the controller or other programmable apparatus provide processes for implementing the functions/acts specified in the flowchart and/or block diagram block or blocks.

The following description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with each claim's language, wherein reference to an element in the singular is not intended to mean “one and only one” unless specifically so stated, but rather “one or more.” All structural and functional equivalents to the elements of the various embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As will be appreciated by one skilled in the art, the present invention may be embodied as a method, system, or computer program product. Accordingly, the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present invention may take the form of a computer program product on a computer-usable storage medium having computer-usable program code embodied in the medium.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and “comprising,” when used in this specification, specify the presence of stated features, steps, operations, elements, and/or components, but do not preclude the presence or addition of one or more other features, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. Aspects of the invention were chosen and described in order to best explain the principles of the invention and the practical application, and to enable others of ordinary skill in the art to understand the invention for various embodiments with various modifications as are suited to the particular use contemplated.

What is claimed is:

1. An enclosure comprising
a bottom comprising a first rigid support structure that is impervious to ethanol vapor, a width of the bottom that is at least as wide as a belly diameter of a barrel, and a length of the bottom that is at least as long as a length of the barrel;
a top comprising a second rigid support structure that is impervious to ethanol vapor, a length of the top that is at least as long as the length of the barrel, and a width of the top that is at least as wide as the belly diameter of the barrel;
a net comprising a perimeter of flexible material that is impervious to ethanol vapor, wherein the perimeter of the net is formed on the top and extends orthogonally from the top to the bottom for at least a distance equal to the belly diameter of the barrel; and
a cooling coil positioned between the top and the bottom and distanced at least the belly diameter of the barrel from the bottom.

2. The enclosure of claim 1, wherein the cooling coil is in fluid communication with a container of refrigerant and is in thermal communication with the refrigerant.

3. The enclosure of claim 2, wherein
the cooling coil comprises an intake at a first end of the cooling coil and a return at the second end of the cooling coil; and
wherein the cooling coil receives the refrigerant from the container via an intake and returns a refrigerant to the container via a return.

4. The enclosure of claim 1, further comprising:
the barrel positioned on the bottom and positioned within the net; and
wherein the cooling coil is positioned such that a low point of the cooling coil is positioned over the barrel.

5. The enclosure of claim 1, wherein the net is detachably engaged to the bottom.

6. The enclosure of claim 1, wherein the net comprises a breakable perimeter.

7. The enclosure of claim 1, further comprising:
an atmospheric balancing tube comprising a transition and a body;
wherein the transition comprises a first end and a second end and the body comprises a third end and a fourth end;
wherein the first end of the transition of the atmospheric balancing tube is connected to the bottom flange and extends orthogonally away from the bottom flange of the enclosure;
wherein the second end of the transition connects with the third end of the body;
wherein the body of the atmospheric balancing tube extends away from the transition and extends at least to the top of the enclosure.

8. The enclosure of claim 1, wherein a distance between a closest point on a surface of the cooling coil and a closest point on a surface of a belly of the barrel is substantially one centimeter or less.

9. The enclosure of claim 1, wherein a distance between a closest point on a surface of the cooling coil and a closest point on a surface of a belly of the barrel is substantially one millimeter or less.

10. The enclosure of claim 1, wherein a temperature of the refrigerant in the container is controlled to be between 1.4° C. and 7.4° C.

11. The enclosure of claim 1, wherein a cooling coil body is positioned over the barrel.

12. The enclosure of claim 1, further comprising:
an ethanol vapor concentration detector comprising:
an emitter configured to emit radiation comprising a wavelength of light;
a receiver configured to receive the emitted light;
an output value corresponding to an intensity of light received by the receiver;
wherein the receiver is positioned a predetermined distance from the emitter;
a controller configured to calculate a transmittance of light based on an intensity of light emitted, an intensity of light received, and the known distance between the emitter and the receiver; and
wherein the controller is configured to receive the output value and to adjust a temperature of the cooling coil based on the output value.

13. The enclosure of claim 1, further comprising:
a drip pan, the drip pan comprising a base and a perimeter connected with the base and extending orthogonally away from the base, located under the cooling coil positioned such that the drip pan opens toward the cooling coil.

14. The enclosure of claim 13, further comprising:
a reinsertion hose comprising a first end in the barrel and a second end in the drip pan; wherein the second end of the reinsertion hose is positioned near the bottom of the drip pan.

15. A method of condensing ethanol comprising:
placing a barrel containing alcohol within an enclosure, wherein the enclosure is impervious to ethanol vapor; and
placing a cooling coil over the barrel within the enclosure.

16. The method of claim 15, further comprising:
chilling a refrigerant to a predetermined temperature;
pumping the refrigerant through the cooling coil;
condensing ethanol vapor in the enclosure onto the cooling coil; and
dripping the condensed ethanol onto the barrel.

17. The method of claim 15, further comprising:
detecting a temperature of a refrigerant in a container;
chilling the refrigerant when the temperature is above a predetermined high temperature;
pumping the refrigerant from the container through the cooling coil; and
dripping ethanol condensate onto the barrel.

18. The method of claim 17,
wherein the predetermined high temperature is 7.4° C.

19. The method of claim 15, further comprising:
chilling a refrigerant to a predetermined temperature;
pumping the refrigerant through the cooling coil;
condensing ethanol onto the cooling coil; and
returning the condensed ethanol into the barrel.

20. The method of claim 15, further comprising:
chilling a refrigerant to a predetermined temperature;
pumping the refrigerant through the cooling coil;
condensing ethanol onto the cooling coil; and
receiving the condensed ethanol in a drip pan.

21. The method of claim 20, further comprising:
bottling the condensed ethanol;
labeling the bottled condensed ethanol with the term “angel's share;” and
distributing the labeled condensed ethanol.

22. The method of claim 15, further comprising:
chilling a refrigerant to a predetermined temperature;
detecting an amount of vapor in a volume of the enclosure;
pumping chilled refrigerant through the cooling coil when the amount of vapor reaches a predetermined high amount of vapor; and
condensing ethanol onto the cooling coil.

23. A system comprising:
a container of chilled refrigerant;
an enclosure for a barrel comprising a bottom, a net, a cooling coil, and a top;
the bottom comprising a first rigid support structure that is impervious to ethanol vapor, a width of the bottom that is at least twice as wide as a belly diameter of a barrel, and a length of the bottom that is at least as long as a length of the barrel;
the top comprising a second rigid support structure that is impervious to ethanol vapor, a length of the top that is at least as long as the length of the barrel, and a width of the top that is at least as wide as the belly diameter of the barrel;
the net comprising a flexible material that is impervious to ethanol vapor, wherein the net forms a perimeter on the top and the perimeter of the net extends orthogonally from the top to the bottom for at least a distance equal to the belly diameter of the barrel;
the cooling coil positioned between the top and the bottom and distanced at least the belly diameter of the barrel from the bottom; and
wherein the cooling coil is in fluid communication with the container of chilled refrigerant and is in thermal communication with the chilled refrigerant.
